(19) United States
(12) Patent Application Publication
(10) Pub. No.: US 2004/0005327 A1
(43) Pub. Date: Jan. 8, 2004

(54) Immune T-cell stimulation

(76) Inventors: Subramanian Iyer, Hockessin, DE (US); Ruye Xing, Avondale, PA (US)

Correspondence Address: RATNERPRESTIA, P.O. BOX 1596, WILMINGTON, DE, 19899, US

(21) Appl. No.: 10/439,162

(22) Filed: May 15, 2003

Related U.S. Application Data
(60) Provisional application No. 60/381,570, filed on May 16, 2002.

(51) Int. Cl. A61K 39/00; A61K 39/38

(52) U.S. Cl. 424/184.1

(57) ABSTRACT

The present invention is a novel approach for stimulating or enhancing immune T-cells in an animal. In particular, the invention is directed to an egg, and more specifically, a fraction thereof of a size less than 3000 daltons, obtained from an avian that has been hyperimmunized with one or more immunogens. The ingestion by an animal of an effective amount of the hyperimmune egg or fraction thereof causes a significant increase in the overall T-cell population in that animal.

### RELATED APPLICATIONS

This application claims priority from the U.S. Provisional Application No. 60/381,570, filed May 16, 2002.

### FIELD OF THE INVENTION

The invention relates to a method for stimulating or enhancing immune T-cell activity in a subject animal using a naturally occurring composition. More particularly, the invention relates to the administration of an egg, or fraction thereof, obtained from a hyperimmunized avian to stimulate or enhance activity of immune T-cells in that animal.

### BACKGROUND OF THE INVENTION

The stimulation and enhancement of T-cells is of great importance to the generation of an immune response, the survival of mature T-cells and thymocyte development. It is an important part of the specific acquired immune system that eradicates cells infected with microorganisms. It also forms the basis of the host's ability to recognize self. T-cells undergo positive and negative selection for the ability to recognize self: MHCp complexes without becoming fully activated, thereby preventing an immune response to host tissues.

The stimulation of T-cells could enhance Cytotoxic T lymphocyte (CTL) population, which have the capacity to lyse target cells, an important effector activity of T lymphocytes. Stimulation of human T-cells is a powerful method to obtain populations of T-cells that are cytotoxic towards autologous tumor cells. It can also augment helper T lymphocytes, which are involved in the activation and immunoglobulin production of human B lymphocytes.

The antigen-specific immune response of naive T-cells involves a very complex set of coordinated events. This activation induces the differentiation, the proliferation and the effector functions of T-cells. Various parameters have been identified which together govern this differentiation process such as antigen route and dose, microenvironment, antigen presenting cells (APC) and the nature of the antigen. The initial critical events of immune T cell activation result from the interaction of specific T cell receptor (TCR) on the T cell and the antigenic peptide bound to MHC molecules on the APC. These events are critical for the production of cytokines such as IL-2 as well as for the expression of cytokine receptors. This, in turn, will determine the extent and duration of T cell proliferation. The regulation of IL-2 production by activated T-cells is one of the key features of the regulation of T cell activation.

Various genera of the class Aves, such as chickens (gallus domesticus), turkeys, and ducks, produce antibodies in blood and eggs against immunogens that cause avian diseases, as well as against other immunogens. For example, LeBacq-Verheyden et al. (Immunology 27:683 (974)) and Leslie, G. A., et al. (J. Med. 130:1337 (1969)), have quantitatively analyzed immunoglobulins of the chicken. Poison et al. (Immunological Communications 9:495-514 (1980)) immunized hens against several proteins and natural mixtures of proteins, and detected IgY antibodies in the yolks of the eggs. Fertel et al. (Biochemical and Biophysical Research Communications 102:1028:1033 (1981)) immunized hens against prostaglandins and detected antibodies in the egg yolk. Jensenius et al. (Journal of Immunological Methods 46:63-68 (1981)) provide a method of isolating egg yolk IgG for use in immunodiagnostics. Poison et al. (Immunological Communications 9:475-493 (1980)) describes antibodies isolated from the yolk of hens that were immunized with a variety of plant viruses.

Hyperimmunized eggs have been developed and have been shown to over-produce antibodies and certain biological factors. Examples of some of these developments are as follows:

U.S. Pat. No. 4,357,272 discloses the isolation of antibodies from the yolks of eggs derived from hyperimmunized hens. The antibody response was elicited by repetitive injections of immunogens derived from plant viruses, human IgG, tetanus antitoxin, snake antivenins, and Serameba.

U.S. Pat. No. 4,550,019 discloses the isolation from egg yolks of antibodies raised in the hen by hyperimmunization with immunogens having a molecular or particle weight of at least 30,000. The immunogens used to hyperimmunize the chickens were selected from among plant viruses, human immunoglobulins, tetanus toxin, and snake venoms.

U.S. Pat. No. 4,748,018 discloses a method of passive immunization of a mammal that comprises parenterally administering purified antibody obtained from the eggs of an avian that has been immunized against the corresponding antigen, and wherein the mammal has acquired immunity to the eggs.

U.S. Pat. No. 5,772,999, discloses a method of preventing, countering or reducing chronic gastrointestinal disorders or Non-Steroidal Anti-Inflammatory Drug-induced (NSAID-induced) gastrointestinal damage in a subject by administering hyperimmunized egg and/or milk or fractions thereof to the subject.

U.S. Pat. No. 6,420,337 discloses a novel Cytokine Activating Factor (CAF), isolated from a hyperimmunized egg, that up regulates the expression of certain pro-inflammatory cytokines, including TNF∝, IL-6 and IL-1β, and down regulates TGFβ.

None of these references, however, discloses or suggests that eggs or fractions thereof, when administered to animals, have the capability to stimulate or enhance immune T-cell activity in that animal. Nor do these references disclose or suggest a method providing a reasonable expectation that hyperimmunization of an avian with a non-specific vaccine could induce an avian to lay eggs having such a capability when administered to a subject animal.

### SUMMARY OF THE INVENTION

It is an object of the invention to provide a method for stimulating production of immune T-cells in an animal by administering to said animal an effective amount of an egg product.

It is a further object of the invention to provide a method for enhancing production of immune T-cells in an animal by administering to said animal an effective amount of an egg product.

### BRIEF DESCRIPTION OF THE DRAWINGS

FIG. 1 is a line graph depicting the stimulation index of Jurkat cells that were incubated over varying periods of time with the less than 3000 dalton fraction of the hyperimmune egg.

FIG. 2 is a line graph depicting the stimulation index of Daudi cells that were incubated over varying periods of time with the less than 3000 dalton fraction of the hyperimmune egg.

### DETAILED DESCRIPTION OF THE INVENTION

The present invention uses a novel approach for stimulating or enhancing activity of immune T-cells in an animal. The invention comprises a hyperimmune egg, or fraction thereof, obtained from an avian that has been hyperimmunized with one or more immunogens. The ingestion by an animal of an effective amount of the hyperimmune egg or fraction thereof causes a significant stimulation of the T-cell population in that animal.

The egg product of the invention is a particularly attractive product because it is completely natural, and, as such, can be administered to stimulate or enhance T-cell activity in an animal without the fear of the side effects that are generally associated with many of the presently available immune-stimulating products. Clearly those allergic to eggs or having an intolerance to eggs may not be able to ingest the hyperimmune egg product in certain administerable forms.

It is the inventors' belief that the beneficial properties referred to above (i.e. T-cell stimulation and enhancement) is due to certain immune factors that are elicited or enhanced via the hyperimmunization process. These immune factors are not believed to be antibodies because, as set forth in the Examples, a fraction of the hyperimmune egg of a size less than 3000 daltons was more effective than the whole egg itself in stimulating and enhancing immune T-cell activity. Clearly a fraction less than 3000 daltons in size does not contain antibodies. Therefore, it is the inventors belief that these factors are instead some type of immune modulating factors that effectively induce the cellular arm of the immune response, causing a stimulation of immune T-cells.

Definitions:

The terms “egg” or “whole egg” each mean any whole egg, whether table egg, hyperimmune egg or otherwise.

The terms “egg product” or “fraction thereof” each mean any product or fraction derived from an egg.

The terms “table egg” or “table egg product” or “fraction thereof” each mean a whole egg, or any product or fraction derived therefrom, obtained from egg-producing animals which are not maintained in a hyperimmune state.

The terms “hyperimmune egg” or “hyperimmune egg product” or “fraction thereof” each mean whole egg or any product or fraction derived therefrom, obtained from an egg producing animal maintained in a hyperimmune state.

The term “immunogen” means a substance that is able to induce a humoral antibody and/or cell-mediated immune response and react with the products of it, e.g., antibody.

The term “immune modulating factor” means a substance, other than an antibody, that affects the immune system.

The term “combinatorial derived immunogens” refers to a novel process of generating molecular diversity among immunogens by way of combinatorial synthesis.

The term “bioengineered immunogens” refers to immunogens which are obtained through the process of gene cloning technologies and genetic manipulation which allow the insertion of encoding nucleotides which can give rise to epitopes having immunogenic properties.

The term “genetic vaccine” refers to a nucleic acid vaccine which is generally produced by recombinant technologies and which may elicit an immune response.

The term “administer” means any method of providing a subject with a substance, including orally, intranasally, intraoptically, parenterally (intravenously, intramuscularly, or subcutaneously), rectally or topically. The term “animal” means the animal kingdom definition.

The term “target animal” refers to an animal which functions as the egg or egg product producing animal.

The term “subject animal” refers to the animal which is administered the egg or egg product produced by the target animal.

“T-cell” or “T-lymphocyte” means a type of lymphocyte that matures in the thymus gland and has an important role in the immune response. There are several subclasses: killer T-cells are responsible for killing cells that are infected by a virus; helper T-cells induce other cells (B-lymphocytes) to produce antibodies

Preparation of Hyperimmune Egg Product

It is important to keep in mind that the hyperimmunization process can be performed with either avians, whereby the egg contains the beneficial elements resulting from the hyperimmunization process, or with bovine whereby the milk contains the beneficial elements resulting from the hyperimmunization process. The following description is limited to hyperimmunization of avians, although it is contemplated that the same concept is applicable to the hyperimmunization of bovine and the collection of their hyperimmune milk.

The hyperimmune egg product can be produced by any egg-producing animal. It is preferred that the animal be a member of the class Aves or, in other words, an avian. Within the class Aves, domesticated fowl are preferred, but other members of this class, such as turkeys, ducks, and geese, are a suitable source of hyperimmune egg product.

The hyperimmune egg product is provided as a spray dried egg powder and is obtained from laying hens vaccinated with a panel of human enteric pathogens (see Example 1). It is submitted that any immunogen or collection of immunogens can be used in the hyperimmunization process of this invention. The process of spray drying the pasteurized liquid egg minimizes damage to the antibodies and immune modulating factors in the egg, resulting in a product that has a high nutrient value and is capable of conferring passive protection to opportunistic enteric infections and appears capable of decreasing inflammation. Antibodies, as a group, are especially resistant to destruction by normal enzymes, and upon oral consumption, a significant fraction will pass through the gastrointestinal tract intact and active. Numerous studies report that orally consumed antibodies offer protection against specific enteric agents.

Further, when such egg-producing animals are brought to a specific state of immunization by means of, for example, periodic booster administrations of antigens, the animals will produce eggs that, when consumed by a subject animal, will have beneficial properties which stimulate the increase of T-cells in the subject animal.

Having knowledge of the requirement for developing and maintaining a hyperimmune state, it is within the skill of the art to vary the amount of immunogen administered, depending on the egg-producing animal genera and strain employed, in order to maintain the animal in the hyperimmune state.

The hyperimmune state is preferably produced by any immunogen or combination of immunogens. Hyperimmunization is preferably achieved by multiple exposures to multiple immunogens, multiple exposures to single immunogens, or single exposures to libraries of immunogens.

In addition to immunizations with naturally occurring immunogens, immunization may also be accomplished using immunogens which are synthetically derived by combinatorial chemistries. The basic strategy is to assemble multiple combinations of chemical building blocks for producing a population of molecules with diversity. Several methods have recently been developed for solid and solution phase combinatorial synthesis of libraries of oligomers (Fodor, S. et al., Science 251:767 (1991); Houghton, R. et al., Nature 354:82 (1991) as well as small organic molecules (Bunin, B. & Ellman, J., J. Am. Chem. Soc. 114:10997 (1992)). Rapid multiple peptide and oligomer synthesis can serve as a source for combinatorial derived immunogens. Furthermore, an alternative strategy would allow the addition of organic building blocks in combinatorial fashion to a backbone molecule for improved immunogenicity.

Alternative modes of hyperimmunizing egg producing animals can be used which, in place of immunogenic vaccines, include the use of genetic vaccines. In particular, any DNA construct (generally consisting of a promoter region and an immunogen encoding sequence) will trigger an immune response. Genetic vaccines consist of immunogen-coding vectors, fragments of naked DNA, plasmid DNA, DNA-RNA antigens, DNA-protein conjugates, DNA-liposome conjugates, DNA expression libraries, and viral and bacterial DNA delivered to produce an immune response. Methods of DNA delivery include particle bombardment, direct injection, viral vectors, liposomes and jet injection, among others. When applying these delivery methods, much smaller quantities may be necessary and generally result in more persistent immunogen production. When using such genetic processes, the preferred method for introducing DNA into avians is through intramuscular injection of the DNA into the breast muscle.

Preferred Hyperimmunization Procedure:

The following list of steps is an example of a preferred procedure used to bring an egg-producing animal to a heightened state of immunity:

1. Selecting one or more immunogens.

2. Eliciting an immune response in the egg-producing animal by primary immunization.

3. Administering booster vaccines of immunogens of appropriate dosage to induce and maintain the hyperimmune state.

Step 1: Any immunogens or combination of immunogens may be employed as a vaccine. The immunogens can be bacterial, viral, protozoan, fungal, cellular, or any other substances to which the immune system of an egg-producing animal will respond. The critical point in this step is that the immunogen(s) must be capable of inducing immune and hyperimmune states in the egg-producing animal. Although only a single antigen may function as the vaccine for the method of the invention, one preferred vaccine is a mixture of polyvalent bacterial and viral antigens selected from the following antigen families: the enteric bacilli and bacteroides, Pneumococci, Pseudomonas, Salmonella, Streptococci, Bacilli, Staphylococci, Neisseria, Clostridia, Mycobacteria, Actinomycetes Chlamydiae, and Mycoplasma. Viral antigens are preferably selected from the following antigen families: adenoviruses, picornaviruses and herpes viruses, although other viral antigen families will work.

In a specifically preferred embodiment, a polyvalent vaccine referred to as PL-100 is used. The bacteria included in the PL-100 vaccine are listed in table 1 of Example 1.

Step 2: The vaccine can be either a killed or live-attenuated vaccine and can be administered by any method that elicits an immune response. It is preferred that immunization be accomplished by administering the immunogens through intramuscular injection. The preferred muscle for injection in an avian is the breast muscle. Other methods of administration that can be used include intravenous injection, intraperitoneal injection, intradermal, rectal suppository, aerosol or oral administration. When DNA techniques are used for the hyperimmunization process, much smaller quantities are required, generally 1-100 micrograms.

It can be determined whether the vaccine has elicited an immune response in the egg-producing animal through a number of methods known to those having skill in the art of immunology. Examples of these include enzyme-linked immunosorbent assays (ELISA), tests for the presence of antibodies to the stimulating antigens, and tests designed to evaluate the ability of immune cells from the host to respond to the antigen. The minimum dosage of immunogen necessary to induce an immune response depends on the vaccination procedure used, including the type of adjuvants and formulation of immunogen(s) used as well as the type of egg-producing animal used as the host.

Step 3: The hyperimmune state is preferably induced and maintained in the target animal by repeated booster administrations of an appropriate dosage at fixed time intervals. The time intervals are preferably 2-8 week intervals over a period of 6-12 months. Dosage is preferably 0.05-5 milligrams of the immunogenic vaccine. However, it is essential that the booster administrations do not lead to immune tolerance. Such processes are well known in the art.

It is possible to use other hyperimmunization maintenance procedures or combination of procedures, such as, for example, intramuscular injection for primary immunization and intravenous injection for booster injections. Further procedures include simultaneously administering microencapsulated and liquid immunogen, or intramuscular injection for primary immunization, and booster dosages by oral administration or parenteral administration by microencapsulation means. Several combinations of primary and hyperimmunization are known to those skilled in the art.

Processing and Administration Of Hyperimmune Egg

Once the egg-producing animals have been sufficiently hyperimmunized, it is preferred that the eggs from these animals are collected and processed to produce a hyperimmune egg product. Subsequently, the hyperimmune egg product can be administered to the subject.

The egg and/or egg product of the present invention is administered to a subject animal by any means that stimulates or enhances immune T-cell activity in the subject animal. It is preferred that administration be done by directly feeding the egg or any effective fraction thereof. Egg and egg yolk are natural food ingredients and are, as such, non-toxic and safe, other than to those with allergies thereto.

One preferred method for preparing the egg involves drying the egg into an egg powder. Although various methods are known for drying eggs, spray drying is a preferred method. The process of spray drying eggs is well known in the art.

In a preferred embodiment, the hyperimmune egg is administered together with a food product or dietary supplement containing several nutrients such as vitamins and minerals. Along these lines, dried egg powder can also be incorporated into drinks in the form of, for example, protein powders, power building drinks, protein supplements and any other nutritional, athlete-associated products.

In an alternative embodiment, the egg powder can be used in bake mixes, power bars, candies, cookies, etc. Other examples of egg processing include making an omelet, soft or hard-boiling the egg, baking the egg, or, if desired, the egg can be eaten raw or processed as liquid egg.

Finally, it is generally known in the art that the yolk and/or white fractions contain the agent or agents responsible for the beneficial properties observed and referred to above. Those having ordinary skill in the art would clearly recognize that further separation could provide more potent fractions or elimination of undesirable components, and would allow for other modes of administration such as administering egg product parenterally, subcutaneously, intravenously, intramuscularly, intraperitoneally, intranasally, orally or topically. Such further separation will provide for the ability to make encapsulated products and pharmaceutical compositions with said egg or fraction thereof.

It is important to administer to the subject animal an amount of hyperimmune egg product that is immunologically effective in stimulating or enhancing immune T-cells. As described above, it is the fraction of the hyperimmune egg less than 3000 daltons in size (see Example 2 for preparation) that proved to be the most effective. In this regard, the inventors have found that administration of anywhere from 0.1 μg/mL to 1000 mg/mL of the less than 3K fraction of hyperimmune egg per subject animal, depending upon the size and weight of the subject, is effective in stimulating or enhancing T-cells. It is more preferred, however, to administer from about 0.2 μg/mL to about 200 μg/mL and even more preferred, 0.5 μg/mL to 20 μg/mL of the less than 3K fraction of hyperimmune egg per subject animal. Duration and intensity of the treatment will depend upon the particular condition of the subject, whether manifestation of a disease or disorder is present, and, if so, the advancement of the disease or disorder in the subject animal. In this regard, the hyperimmune egg product is administered in an amount that effectively stimulates or enhances T-cell activity in order to strengthen the immune system to battle the disease or condition. It is noted that while the less than 3K fraction of hyperimmune egg is preferred, larger fractions and whole egg are also effective. Daily amounts ranging from less than one to several whole, hyperimmune eggs (or hyperimmune egg products containing the equivalent of less than one to several whole, hyperimmunized eggs) can be administered to the subject depending on the particular condition of the subject animal. More potent fractions can be separated and concentrated by methods well-known in the art.

The advantageous properties of this invention can be observed by reference to the following examples which illustrate the invention.

### EXAMPLES

### Example 1

Preparation of PL-100 Vaccine

The multivalent vaccine referred to as PL-100 and containing the bacteria shown in Table 1 (obtained from the American Type Culture Collection), was reconstituted with 15 ml of medium and incubated overnight at 37C. Once good growth was obtained, approximately one-half of the bacterial suspension was used to inoculate one liter of broth which was then incubated at 37C. The remaining suspension was transferred to sterile glycol tubes and stored at −20C. for up to six months.

After good growth was visible in the culture, the bacteria were harvested by centrifugation. The bacterial pellet was resuspended in sterile physiological saline solution and the bacterial sample was centrifuged three times to wash the cells. After the third wash, the pellet obtained was resuspended in a small amount of double distilled water.

The medium-free bacterial suspension was heat-killed by placing the suspension in a glass flask in an 80C. water bath overnight. The viability of the broth culture was tested with a small amount of heat-killed bacteria. Broth was inoculated with heat-killed bacteria, incubated at 37C. for five days and checked daily for growth, as the bacteria have to be killed for use in the vaccine.

The heat-killed bacteria were lyophilized until dry. The dry bacteria were then mixed with sterile saline solution to a concentration of 2.2×10⁸ bacterial cells/ml saline (1.0 optical density reading at 660 nm).

TABLE 1
Antigens in the PL-100 Vaccine

| Name | Media |
|---|---|
| Staphylococcus simulans | BHI |
| Staphylococcus epidermidis | BHI |
| Streptococcus pyogenes, A Type 1 | APT |
| Streptococcus pyogenes, A Type 3 | APT |
| Streptococcus pyogenes, A Type 5 | APT |
| Streptococcus pyogenes, A Type 8 | APT |
| Streptococcus pyogenes, A Type 12 | APT |
| Streptococcus pyogenes, A Type 14 | APT |
| Streptococcus pyogenes, A Type 18 | APT |
| Streptococcus pyogenes, A Type 22 | APT |
| Escherichia coli (ATCC #26) | BHI |
| Escherichia coli (ATCC #884) | BHI |
| Salmonella enteritidis | BHI |
| Pseudomonas aeruginosa | BHI |
| Klebsiella pneumoniae | BHI |
| Salmonella typhimurium | BHI |
| Haemophilus influenzae | BHI |
| Streptococcus mitis | APT |
| Proteus vulgaris | BHI |
| Shigella dysenteriae | BHI |
| Diplococcus pneumoniae | APT |
| Propionibacter acnes | Broth |
| Streptococcus sanguis | APT |
| Streptococcus salivarus | APT |
| Streptococcus mutans | BHI |
| Streptococcus agalactiae | APT |

Immunization Procedure for Hyperimmune Egg Product

A killed preparation of pathogens was prepared as described above. For the first vaccination, the bacteria were mixed with complete Freund's adjuvant, and 5.6 mg of bacterial material were injected into the breast muscle of a chicken. For the remaining vaccines, the bacterial preparation was mixed with the incomplete Freund's adjuvant and injected into the chickens at two week intervals for six months. Eggs were collected from the hyperimmunized chickens and then sprayed dried into a powder form. During the spray drying procedure, inlet temperatures did not exceed 320 Degrees F., exhaust temperatures were maintained in accordance with producing powder in the range of 3.0 to 4.0 percent finished moisture, and pump pressure was maintained around 2500 to 4000 P.S.I. Lower temperatures ranging from 100-160 F. were used, and samples were monitored for moisture contend during the drying process to obtain a final product having any consistency desired.

### Example 2

Preferred Method for Preparing a Partially Purified Fraction of an Egg

The following examples describe a method (suitable for large scale purification) for obtaining a partially purified fraction of egg having a low molecular weight, and being in a non-aggregated form. Whole eggs, hyperimmunized and control table eggs, were cracked and the egg white was separated from the yolk and both were spray-dried. Hyperimmune eggs were obtained as described in Example 1. Egg white powder was processed separately to obtain the aqueous fraction for ultra-filtration.

All of the purification steps were performed so as to minimize possible contamination with bacteria or pyrogens. Sterile water was used to prepare solutions and all glassware was de-pyrogenated. In addition, the solution was sterile filtered.

Preparation from Egg Yolk

Solvent Extraction

The dried egg yolk as prepared in Example 1, was subjected to liquid solvent extraction with either propane, or butane to separate the lipids from the aqueous yolk fraction. Briefly, 500 grams of dry egg yolk powder was placed in a column, to which was added 4 liters of liquid propane solvent. The solvent supernatant and extracted lipid were removed. Six additional solvent extractions were performed for a total of six lipid extractions.

Ultra-Filtration

Four hundred grams of dry de-fatted egg yolk was diluted with 4 liters of sterile distilled water and homogenized with a Virtis (handishear). The yolk mixture was either centrifuged at 24 RPM or allowed to stand refrigerated until the non-dissolved yolk particles precipitated. The resulting aqueous fraction was ultrafiltered using an Amicon RA1000 ultra-filtration system equipped with 3,000 dalton cut-off spiral-wound membrane. The pump speed was maintained at 20 psi inlet pressure and 15 psi outlet pressure. The <3,000 daltons molecular weight permeates was sterile filtered using a 0.45 μm Sterile disposable Nalgene filter and lyophilized or frozen, for storage, bioassay testing or further purification.

Molecular species below 3,000 daltons from egg yolk comprise the partially purified egg fraction having a low molecular weight and being in a non-aggregated form. From 400 grams of starting material, the yield of the <3K dalton partially purified fraction was approximately 12 grams or 3% of the total.

Preparation from Egg White

Four hundred grams of egg white, isolated from both hyperimmunized egg as described in Example 1 and control table egg, was diluted with four liter of deionized water. The mixture was homogenized and filtered through a 40 μm filter and ultrafiltered through a 3 KDa MW CO ultra-filtration system. From 400 g of egg white powder, 8.6 g or 2.15% of the less than 3K dalton partially purified egg fraction was recovered.

### Example 3

Immune Stimulatory Activity in Immune Cell Lines

All cell lines, obtained from ATCC, were grown in their recommended media. These cells were grown in a 96 well plate and allowed to reach exponential growth phase. The 3k fraction was appropriately diluted, and added to the respective wells and incubated for 24-96 hours. Viability was determined by staining the cells using Alamar Blue, and reading the fluorescence on an automated microplate reader at an excitation wavelength of 520 nm. The stimulation index (SI) represents the relative number of cells in the treated versus that in the unstimulated control group.

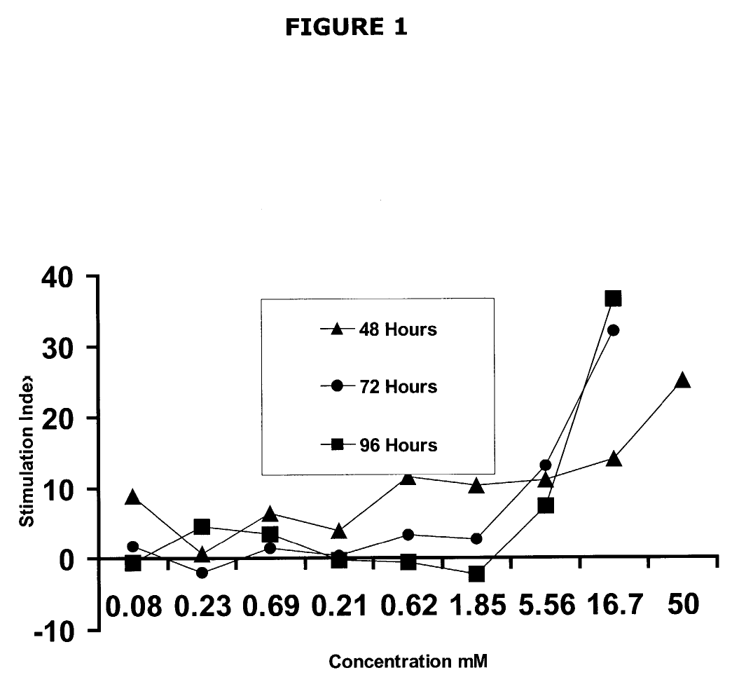

FIG. 1 is a graph showing the Stimulation Index (SI) of Jurkat cells (a T-cell Leukemia cell line) that were incubated for varying periods (from 24-96 hours) with the less than 3K fraction of the hyperimmune egg. At a concentration range of 5.5 - 50 μM there is a marked increase in the SI, which denotes hyperstimulation of the Jurkat cells. This is consistent in the 48, 72 and 96 hr time period of cell activation.

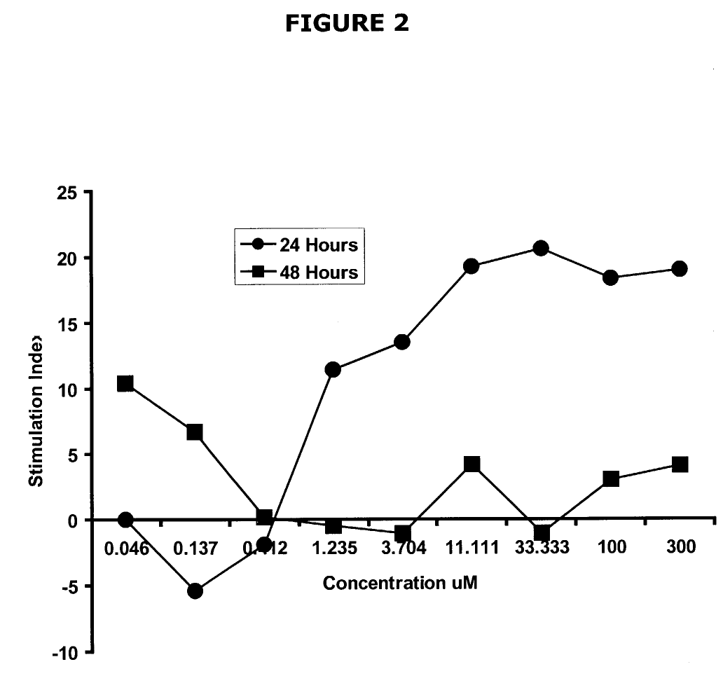

FIG. 2 is a graph showing the Stimulation Index (SI) of Daudi cells (a B lymphoblastoid cell line), that were incubated for varying periods (from 24-48 hours) with the 3k fraction of the hyperimmune egg. Results indicate that the hyperstimulation occurs at 24 hours in the 1.24-300 μM concentration of the 3k fraction.

Thus, the results from the effect of the 3k fraction of the hyperimmune egg on Jurkat and Daudi cell lines, indicates strong immune stimulatory activity. This prompted us to perform the ³H-Thymidine incorporation experiments in PHA mediated T cell stimulation assays, as set forth in Example 4 below.

### Example 4

In Vitro Proliferation of Peripheral Lymphocytes Treated with PHA

The test was carried out using peripheral lymphocyte cultures, obtained from healthy voluntary donors. Lymphocytes were treated with different concentrations of PHA and their growth kinetics was compared with the one presented by the cells plus Salt Saline Solution.

As a result, a mitogenic effect was observed on Peripheral Lymphocytes in relation with the control (Peripheral Lymphocytes plus Salt Saline Solution) with low concentrations that include 0.1, 0.5, 1.0 and 2.5%. The Stimulating Index, corresponding to those concentrations, are 1.6, 3.02, 2.83 and 1.8, and this means that in the test conditions, there was a cell population increasing of 160, 302, 283 and 180% respectively.

Blastogenesis of T-cells

Dilutions of PHA were prepared from 1:100 to 1:1000 and the dilutions were added to mycro-plate system lymphocyte cultures. Phytohemaglutinin, Pockeweed antigen and A Concanavalin were added to identified wells. After the culture period, T-lymphocytes proliferation was determined by radioactive tymidine uptake assay.

PHA increased the proliferation of free mitogen lymphocytes intensively.

The present study compared the mitogenic effect of hyperimmune whole egg and a partially purified fraction of the hyperimmune egg (less then 3K fraction described in Example 2) against control medium alone. The hyperimmune whole egg was tested at varying concentrations that include 0.24, 0.98, 3.9, 15.6, 62.5 and 250 μg/ml. The purified less than 3K fraction from the hyperimmune egg was tested at 0.1, 0.2, 0.7, 2.0, 6.1, 18.4, 55.3 and 166.0 μg/ml concentrations. The Stimulating Index for hyperimmune whole egg, in accordance with the above concentrations, was 0.9, 0.8, 0.9, 0.9, 0.8, and 0.8 respectively, which corresponds, in the test conditions, to a cell population increase of 90, 80, 90, 90, 80 and 80% respectively. The Stimulating Index for the 3K purified fraction, in accordance with the above concentrations, was 1.1, 1.2, 1.5, 1.7, 1.4, 1.3, 1.0 and 1.2 respectively (see Table 2), which corresponds, in the test conditions, to a cell population increasing of 110, 120, 150, 170, 140, 130, 100 and 120% respectively.

These results indicate that the whole hyperimmune egg, at concentrations ranging from 0.24-250 μg/ml does not significantly stimulate the T-cells in vitro. The less than 3K purified fraction of the hyperimmune egg, on the other hand, showed significant stimulation of the T-cells at 0.7 μg/ml-20.0 μg/ml. Table 1 shows the actual number of cells that were stimulated per well.

This activity of the purified fraction of the hyperimmune egg could be due to the cytokine activating factors that could specifically induce the cytokines involved in stimulating the cellular arm of the immune system.

TABLE 1
3H-thymidine incorporation/well [cpm]

| | PHA | | | | | |
|---|---|---|---|---|---|---|
| | [μg/ml] | Control | <3K Fraction [μg/ml] | | | |
| No. | 5 | 0 | 166.00 | 55.33 | 18.44 | 6.15 |
| 1 | 101241 | 541 | 890 | 624 | 819 | 958 |
| 2 | 111452 | 714 | 765 | 578 | 637 | 759 |
| 3 | 98898 | 552 | 648 | 649 | 711 | 759 |
| 4 | 121212 | 633 | 705 | 600 | 967 | 697 |
| 5 | 97149 | 465 | 506 | 523 | 723 | 604 |
| 6 | 118218 | 406 | 437 | 436 | 475 | 813 |
| AVE | 108028 | 552 | 659 | 568 | 722 | 765 |
| Neg | 10365 | 111 | 167 | 78 | 166 | 118 |
| | PHA | | | | | |
| | [μg/ml] | Control | <3K Fraction [μg/ml] | | | |
| No. | 5 | 0 | 2.05 | 0.68 | 0.23 | 0.08 |
| 1 | 101241 | 541 | 1050 | 806 | 754 | 644 |
| 2 | 111452 | 714 | 890 | 689 | 794 | 517 |
| 3 | 98898 | 552 | 1010 | 944 | 639 | 688 |
| 4 | 121212 | 633 | 806 | 698 | 573 | 636 |
| 5 | 97149 | 465 | 1009 | 809 | 494 | 667 |
| 6 | 118218 | 406 | 1009 | 919 | 802 | 393 |
| AVE | 108028 | 552 | 962 | 811 | 676 | 591 |
| Neg | 10365 | 111 | 94 | 107 | 127 | 114 |

TABLE 2
Stimulation Index

| | PHA | | | | | |
|---|---|---|---|---|---|---|
| | [μg/ml] | Control | PL-100 [μg/ml] | | | |
| No. | 5 | 0 | 166.00 | 55.33 | 18.44 | 6.15 |
| 1 | 183.5 | 1.0 | 1.6 | 1.1 | 1.5 | 1.7 |
| 2 | 202.0 | 1.3 | 1.4 | 1.0 | 1.2 | 1.4 |
| 3 | 179.2 | 1.0 | 1.2 | 1.2 | 1.3 | 1.4 |
| 4 | 219.7 | 1.1 | 1.3 | 1.1 | 1.8 | 1.3 |
| 5 | 176.0 | 0.8 | 0.9 | 0.9 | 1.3 | 1.1 |
| 6 | 214.2 | 0.7 | 0.8 | 0.8 | 0.9 | 1.5 |
| AVE | 195.8 | 1.0 | 1.2 | 1.0 | 1.3 | 1.4 |
| Neg | 18.8 | 0.2 | 0.3 | 0.1 | 0.3 | 0.2 |
| | PHA | | | | | |
| | [μg/ml] | Control | PL-100 [μg/ml] | | | |
| No. | 5 | 0 | 2.05 | 0.68 | 0.23 | 0.08 |
| 1 | 183.5 | 1.0 | 1.9 | 1.5 | 1.4 | 1.2 |
| 2 | 202.0 | 1.3 | 1.6 | 1.2 | 1.4 | 0.9 |
| 3 | 179.2 | 1.0 | 1.8 | 1.7 | 1.2 | 1.2 |
| 4 | 219.7 | 1.1 | 1.5 | 1.3 | 1.0 | 1.2 |
| 5 | 176.0 | 0.8 | 1.8 | 1.5 | 0.9 | 1.2 |
| 6 | 214.2 | 0.7 | 1.8 | 1.7 | 1.5 | 0.7 |
| AVE | 195.8 | 1.0 | 1.7 | 1.5 | 1.2 | 1.1 |
| Neg | 18.8 | 0.2 | 0.2 | 0.2 | 0.2 | 0.2 |

The same study was repeated. Below, in Tables 3 and 4 are the results. Similar increases in T-cell stimulation were observed.

TABLE 3
3H-thymidine incorporation/well [cpm]

| | PHA | | | | | |
|---|---|---|---|---|---|---|
| | [μg/ml] | Control | <3K Fraction [μg/ml] | | | |
| No. | 5 | 0 | 166.00 | 55.33 | 18.44 | 6.15 |
| 1 | 102484 | 335 | 461 | 548 | 361 | 526 |
| 2 | 114522 | 326 | 391 | 461 | 401 | 408 |
| 3 | 110201 | 454 | 501 | 393 | 635 | 494 |
| 4 | 121811 | 397 | 494 | 521 | 344 | 405 |
| 5 | 97453 | 391 | 372 | 493 | 537 | 459 |
| 6 | 112934 | 354 | 353 | 358 | 377 | 396 |
| AVE | 109901 | 376 | 429 | 462 | 443 | 448 |
| NEG | 8746 | 48 | 65 | 74 | 117 | 54 |

| | | |
|---|---|---|
| PHA | | |
| [μg/ml] | Control | <3K Fraction [μg/ml] |

| No. | 5 | 0 | 2.05 | 0.68 | 0.23 | 0.08 |
|---|---|---|---|---|---|---|
| 1 | 102484 | 335 | 537 | 690 | 597 | 619 |
| 2 | 114522 | 326 | 391 | 551 | 584 | 629 |
| 3 | 110201 | 454 | 533 | 433 | 524 | 500 |
| 4 | 121811 | 397 | 477 | 538 | 514 | 807 |
| 5 | 97453 | 391 | 393 | 484 | 474 | 560 |
| 6 | 112934 | 354 | 397 | 508 | 519 | 519 |
| AVE | 109901 | 376 | 455 | 534 | 535 | 606 |
| NEG | 8746 | 48 | 70 | 87 | 46 | 111 |

TABLE 4
Stimulation Index

| | PHA | | | | | |
|---|---|---|---|---|---|---|
| | [μg/ml] | Control | <3K Fraction [μg/ml] | | | |
| No. | 5 | 0 | 166.00 | 55.33 | 18.44 | 6.15 |
| 1 | 272.4 | 0.9 | 1.2 | 1.5 | 1.0 | 1.4 |
| 2 | 304.4 | 0.9 | 1.0 | 1.2 | 1.1 | 1.1 |
| 3 | 293.0 | 1.2 | 1.3 | 1.0 | 1.7 | 1.3 |
| 4 | 323.8 | 1.1 | 1.3 | 1.4 | 0.9 | 1.1 |
| 5 | 259.1 | 1.0 | 1.0 | 1.3 | 1.4 | 1.2 |
| 6 | 300.2 | 0.9 | 0.9 | 1.0 | 1.0 | 1.1 |
| AVE | 292.2 | 1.0 | 1.1 | 1.2 | 1.2 | 1.2 |
| NEG | 23.3 | 0.1 | 0.2 | 0.2 | 0.3 | 0.1 |
| | PHA | | | | | |
| | [μg/ml] | Control | <3K Fraction [μg/ml] | | | |
| No. | 5 | 0 | 2.05 | 0.68 | 0.23 | 0.08 |
| 1 | 272.4 | 0.9 | 1.4 | 1.8 | 1.6 | 1.6 |
| 2 | 304.4 | 0.9 | 1.0 | 1.5 | 1.6 | 1.7 |
| 3 | 293.0 | 1.2 | 1.4 | 1.2 | 1.4 | 1.3 |
| 4 | 323.8 | 1.1 | 1.3 | 1.4 | 1.4 | 2.1 |
| 5 | 259.1 | 1.0 | 1.0 | 1.3 | 1.3 | 1.5 |
| 6 | 300.2 | 0.9 | 1.1 | 1.4 | 1.4 | 1.4 |
| AVE | 292.2 | 1.0 | 1.2 | 1.4 | 1.4 | 1.6 |
| NEG | 23.3 | 0.1 | 0.2 | 0.2 | 0.1 | 0.3 |

Although the invention is illustrated and described herein with reference to specific embodiments, the invention is not intended to be limited to the details shown. Rather, various modifications may be made in the details within the scope and range of equivalents of the claims and without departing from the invention.

1. A method for stimulating immune T-cells in an animal by administering to said animal an effective amount of an egg product.

2. The method of claim 1 wherein the egg product comprises an egg or a fraction of an egg obtained from an avian wherein said avian is hyperimmunized with at least one immunogen.

3. The method of claim 2 wherein the immunogen is selected from the group consisting of the following:
Staphylococcus simulans; Staphylococcus epidermidis;
Streptococcus pyogenes; Escherichia coli; Salmonella enteritidis;
Pseudomonas aeruginosa; Klebsiella pneumoniae;
Salmonella typhimurium; Haemophilus influenzae;
Streptococcus mitis; Proteus vulgaris;
Shigella dysenteriae; Diplococcus pneumoniae;
Propionibacter acnes; Streptococcus sanguis;
Streptococcus salivarus; Streptococcus mutans;
Streptococcus agalactiae:

4. The method of claim 1 wherein the egg product is administered orally.

5. The method of claim 1 wherein the egg product comprises a fraction of a whole egg.

6. The method of claim 5 wherein the fraction comprises a fraction having a size of about 3000 daltons or less.

7. The method of claim 6 wherein the effective amount of the egg product ranges from about 0.1 μg/mL to about 2000 mg/mL.

8. The method of claim 7 wherein the effective amount of the egg product ranges from about 0.2 μg/mL to about 200 μg/mL.

9. The method of claim 8 wherein the effective amount of the egg product ranges from about 0.5 μg/mL to about 20 μg/mL.

10. The method of claim 1 wherein the avian comprises a domesticated fowl.

11. A method for enhancing immune T-cells in an animal by administering to said animal an effective amount of an egg product.

12. The method of claim 11 wherein the egg product comprises an egg or a fraction of an egg obtained from an avian wherein said avian is hyperimmunized with at least one immunogen.

13. The method of claim 12 wherein the immunogen is selected from the group consisting of the following:
Staphylococcus simulans; Staphylococcus epidermidis;
Streptococcus pyogenes; Escherichia coli; Salmonella enteritidis;
Pseudomonas aeruginosa; Klebsiella pneumoniae;
Salmonella typhimurium; Haemophilus influenzae;
Streptococcus mitis; Proteus vulgaris;
Shigella dysenteriae; Diplococcus pneumoniae;
Propionibacter acnes; Streptococcus sanguis;
Streptococcus salivarus; Streptococcus mutans;
Streptococcus agalactiae.

14. The method of claim 11 wherein the egg product is administered orally.

15. The method of claim 11 wherein the egg product comprises a fraction of a whole egg.

16. The method of claim 15 wherein the fraction comprises a fraction having a size of about 3000 daltons or less.

17. The method of claim 16 wherein the effective amount of the egg product ranges from about 0.1 μg/mL to about 2000 mg/mL.

18. The method of claim 17 wherein the effective amount of the egg product ranges from about 0.2 μg/mL to about 200 μg/mL.

19. The method of claim 18 wherein the effective amount of the egg product ranges from about 0.5 μg/mL to about 20 μg/mL.

20. The method of claim 11 wherein the avian comprises a domesticated fowl.
